(19) United States
(12) Patent Application Publication
(10) Pub. No.: US 2007/0112075 A1
(43) Pub. Date: May 17, 2007

(54) Stable pharmaceutical formulations containing escitalopram and bupropion

(75) Inventors: Mahendra G. Dedhiya, Pomona, NY (US); Anil Chhettry, Holtsville, NY (US); Narasimhan Mani, Morris Plains, NJ (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/582,164

(22) Filed: Oct. 16, 2006

Related U.S. Application Data
(60) Provisional application No. 60/727,055, filed on Oct. 14, 2005.

(51) Int. Cl. A61K 31/138

(52) U.S. Cl. 514/651

(57) ABSTRACT

The present invention relates to stable pharmaceutical formulations of escitalopram and bupropion and their use for the treatment a central nervous system disorder, such as a mood disorder (e.g., major depressive disorder) or an anxiety disorder (e.g., general anxiety disorder, social anxiety disorder, post traumatic stress disorder, or panic disorder).

### CROSS-REFERENCES TO RELATED APPLICATIONS

This application claims priority to U.S. provisional patent application Ser. No. 60/727,055 filed Oct. 14, 2005, the contents of which are hereby incorporated by reference in its entirety.

### FIELD OF THE INVENTION

The present invention relates to stable pharmaceutical formulations of escitalopram and bupropion and their use for the treatment of a central nervous system disorder, such as a mood disorder (e.g., major depressive disorder) or an anxiety disorder (e.g., general anxiety disorder, social anxiety disorder, post traumatic stress disorder, or panic disorder).

### BACKGROUND OF THE INVENTION

Selective serotonin reuptake inhibitors (hereinafter called SSRIs), such as racemic citalopram and escitalopram, have become first-choice therapeutics in the treatment of depression primarily due to their superior efficacy compared to tricyclic antidepressants and monoamine oxidase inhibitors (MAOIs). SSRIs function by inhibiting the reuptake of the neurotransmitter serotonin (5-hydroxytryptamine, 5-HT) by nerve cells at synapses. As a result, serotonin persists in the synaptic gap and has the chance to stimulate receptors of recipient cells.

Escitalopram is the S-enantiomer of citalopram and has the following structure: 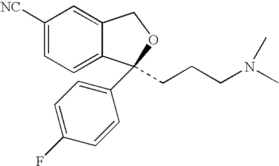
Methods of preparing escitalopram are disclosed in, for example, U.S. Pat. Nos. Re. 34,712 and 6,566,540 and International Publication Nos. WO 03/000672, WO 03/006449, WO 03/051861, and WO 04/083197, all of which are hereby incorporated by reference.

International Publication Nos. WO 01/03694 and WO 02/087566, which are hereby incorporated by reference, disclose the use of escitalopram in the treatment of various mental disorders including major depressive disorder, general anxiety disorder, social anxiety disorder, post traumatic stress disorder, panic attacks, acute stress disorder, eating disorders (such as bulimia, anorexia and obesity), phobias, dysthymia, premenstrual syndrome, cognitive disorders, impulse control disorders, attention deficit hyperactivity disorder and drug abuse. International Publication No. WO 02/087566 also discloses the use of escitalopram for the. treatment of patients who have failed to respond to initial treatment with a conventional SSRI, in particular patients with major depression disorder who have failed to respond to initial treatment with a conventional SSRI.

Escitalopram oxalate is currently marketed in the United States as Lexapro® for the treatment of major depressive disorder and generalized anxiety disorder. Lexapro® is available in 5, 10 and 20 mg escitalopram immediate release tablets (as an oxalate salt) and in a 5 mg/mL oral solution.

A modified release formulation of escitalopram oxalate prepared by melt granulation is disclosed in International Publication No. WO 01/22941. Modified release formulations of SSRIs, such as citalopram hydrobromide and escitalopram oxalate, having particular dissolution profiles are disclosed in International Publication No. WO 2004/058299.

Side effects associated with escitalopram include nausea, insomnia, somnolence, increased sweating, fatigue, and sexual dysfunction (including, but not limited to, ejaculation disorder, anorgasmia, and decreased libido).

Bupropion hydrochloride, which is described in U.S. Pat. Nos. 3,819,706 and 3,885,046, is currently marketed as Wellbutrin®, Wellbutrin SR®, and Wellbutrin XL® for the treatment of major depressive disorder and Zyban® as an aid to smoking cessation treatment. Bupropion is an aminoketone-derivative chemically unrelated to other currently available antidepressants (e.g., selective serotonin-reuptake inhibitors, tricyclics, and tetracyclics). While the neurochemical mechanisms of the antidepressant and smoking cessation effects are unknown, noradrenergic pathways and/or dopaminergic effects appear to be primarily involved. Bupropion does not inhibit monoamine oxidase and is a weak blocker of serotonin and norepinephrine uptake.

Wellbutrin® (an immediate release bupropion hydrochloride formulation) is supplied as 75 and 100 mg tablets which are to be administered three times a day, preferably with at least 6 hours between successive doses. Controlled release formulations of bupropion hydrochloride have been developed.

For example, U.S. Pat. No. Re. 33,994 discloses a controlled release bupropion tablet formulation comprising a bupropion hydrochloride core and a coating comprised of a water-insoluble, water-permneable film forming coating and a particulate, water-soluble, pore-forming material. However, because 25-70% of the bupropion is released within 4 hours and 40-90% within 6 hours, at least twice daily dosing is still typically required.

U.S. Pat. Nos. 5,358,970, 5,763,493, and 5,731,000 disclose bupropion hydrochloride formulations containing a stabilizer to prevent the degradation of the bupropion hydrochloride.

U.S. Pat. No. 5,427,798 discloses a controlled release bupropion tablet formulation containing hydroxypropyl methylcellulose. More than half of the bupropion is preferably released in distilled water in 4 hours. Because of this rapid release rate, the formulation typically is administered multiple times in a day.

U.S. Pat. Nos. 6,096,341 and 6,143,327 disclose a controlled release tablet of bupropion hydrochloride, free of stabilizers and pore-forming agents. The tablet is comprised of a core consisting essentially of bupropion hydrochloride, a binder, and a lubricant, and a coating consisting essentially of a water-insoluble, water-permeable, film-forming polymer, a plasticizer, and a water-soluble polymer.

U.S. Pat. No. 6,905,708 and U.S. Patent Application Publication Nos. 2003/0161874 and 2005/0147678 disclose a once a day bupropion hydrochloride formulation comprising coated pellets of bupropion hydrochloride.

In DeVane, J. Clin. Psychiatry 2003, 64 (suppl. 18):14-19, the results of clinical studies of immediate release and controlled release formulations of antidepressants were compared in relation to nausea leading to drug discontinuation. The author stated that “more stable pharmacokinetic profiles might be the cause for the low occurrence of nausea with some controlled-release newer antidepressants” but a “connection has not been proven.”

According to Gerner et al., Biol. Psychiatry, 1998, 43:101S, abstract 336 (“Gerner I”), “[b]upropion has been added to SSRIs for treatment of inadequate clinical response, SSRI sexual dysfunction, and for comorbid ADD and depression associated with [p]anic or [o]bsessive [c]ompulsive [d]isorders.” See also Kennedy et al. J. Clin. Psychiatry, 2002, 63: 181-186 (study regarding the pharmacokinetic, therapeutic, and sexual dysfunction effects of combinations of bupropion SR with venlafaxine, paroxetine, or fluoxetine); Gerner et al, Biol. Psychiatry, 1998, 43:99S, abstract 329 (“Gerner II”); Ashton et al., J. Clin. Psychiatry, 1998, 59(3):112-115 (study regarding the use of bupropion as an antidote for serotonin reuptake inhibitor (paroxetine, fluoxetine, sertraline, venlafaxine, or fluvoxamine) induced sexual dysfunction); Gitlin et al., J. Sex & Marital Therapy 2002, 28:131-138 (study regarding a bupropion sustained release formulation as a treatment for SSRI-induced sexual side effects). However, treatment of SSRI-induced sexual dysfunction with bupropion has not been proven to be effective. According to Sturpe et al., J. Family Practice August 2002, 51(8):1681, a double-blind placebo-controlled trial comparing augmentation therapy with bupropion showed equal improvement in sexual function to placebo. Furthermore, bupropion has increased seizure incidence compared with other antidepressants. Gerner II, supra (reporting on 3 cases of major motor seizures in previously seizure-free depressed patients after combining bupropion with fluoxetine or fluvoxamine); see also Gerner I, supra.

Studies suggest that between 29% and 46% of depressed patients fail to respond fully with antidepressant treatment of adequate dose and duration. Fava et al., Psychiatr. Clin. North Am., 1996, 19(2):179-200; Fava et al., Ann. Clin. Psychiatry, 2003, 15(1): 17-22. Lam et al., J. Clin. Psychiatry, 2004, 65:337-340, reported the results of a clinical study comparing combining citalopram and bupropion SR versus switching to a monotherapy in patients with treatment-resistant depression. According to the authors, “[t]he results of this cohort study suggest that combining citalopram and bupropion SR is more effective than switching to a monotherapy.”

U.S. Pat. No. 6,342,496 discloses bupropion metabolites for treating disorders ameliorated by inhibition of neuronal monoamine reuptake. The bupropion metabolite can be adjunctively administered with an additional pharmacologically active compound, such as an SSRI, 5-HT₃ inhibitor, or nicotine.

There is a need for once-a-day formulations for treating central nervous system disorders which have fewer side effects than prior formulations and are effective in treatment-resistant patients.

### SUMMARY OF THE INVENTION

The present invention relates to stable oral dosage forms containing escitalopram (or a pharmaceutically acceptable salt thereof) and bupropion (or a pharmaceutically acceptable salt thereof). Preferably, the oral dosage forms are once a day formulations, i.e., only administration once a day is required to provide the patient with a therapeutic effect over the entire day (24 hours). The amount of bupropion or a pharmaceutically acceptable salt thereof in the oral dosage forms preferably ranges from about 50 to about 450 mg and more preferably from about 75 to about 225 mg (calculated based on the weight of a molar equivalent of bupropion hydrochloride) (for example, 75, 150, or 225 mg). The amount of escitalopram or a pharmaceutically acceptable salt thereof in the oral dosage forms preferably ranges from about 2.5 to about 40 mg escitalopram and more preferably from about 2.5 to 20 mg (calculated based on the weight of a molar equivalent of escitalopram free base) (for example, 2.5, 5, 10, or 20 mg). According to one preferred embodiment, the oral dosage form comprises 4 mg of escitalopram or a pharmaceutically acceptable salt thereof and 150 mg of bupropion or a pharmaceutically acceptable salt thereof. The oral dosage form may provide immediate release or modified release of each active component.

Preferably, the bupropion and escitalopram in the oral dosage form are physically separated. The inventors have discovered that conventional formulations of escitalopram hydrobromide and escitalopram oxalate unexpectedly degrade under storage conditions. In particular, escitalopram oxalate, which is stable in commercial formulations up to about 12 months, degrades significantly more rapidly when stored in intimate contact with bupropion hydrochloride. It has been found that when the two are in intimate contact, each degrades by more than 10% in potency after one month of storage at 40° C. and 75% relative humidity. The escitalopram and bupropion may be separated by having separate discrete zones of the dosage form (such as different layers) for each component. Alternatively, the dosage form may include a plurality of escitalopram tablets or beads and a plurality of bupropion tablets or beads, where one or both of the escitalopram tablets/beads and bupropion tablets/beads are coated.

Preferably, the oral dosage form contains (1) at least about 80% w/w of undegraded escitalopram or pharmaceutically acceptable salt thereof (relative to the initial amount of escitalopram or pharmaceutically acceptable salt thereof) after storage for 6 weeks at about 40° C. and 75% relative humidity, (2) at least about 80% w/w of undegraded bupropion or pharmaceutically acceptable salt thereof after storage for 6 weeks at about 40° C. and 75% relative humidity, or both. The oral dosage form more preferably contains at least about 90% w/w and even more preferably 95% w/w of undegraded bupropion or a pharmaceutically acceptable salt thereof and/or undegraded escitalopram or a pharmaceutically acceptable salt thereof after storage for 6 weeks under the same conditions.

According to yet another preferred embodiment, the oral dosage form contains (1) at least about 90% w/w of undegraded escitalopram or pharmaceutically acceptable salt thereof (relative to the initial amount of escitalopram or pharmaceutically acceptable salt thereof) after storage for 1, 3, or 6 months at about 40° C. and 75% relative humidity, (2) at least about 90% w/w of undegraded bupropion or pharmaceutically acceptable salt thereof after storage for 1, 3, or 6 months at about 40° C. and 75% relative humidity, or both. The oral dosage form more preferably contains at least about 95% w/w of undegraded bupropion or a pharmaceutically acceptable salt thereof and/or undegraded escitalopram or a pharmaceutically acceptable salt thereof after storage for 1, 3, or 6 months under the same conditions.

According to another preferred embodiment, the oral dosage form contains (1) at least about 80% w/w of undegraded escitalopram or pharmaceutically acceptable salt thereof (relative to the initial amount of escitalopram or pharmaceutically acceptable salt thereof) after storage for 6 months, 9 months, or 1 year at about 25° C. and 60% relative humidity, (2) at least about 80% w/w of undegraded bupropion or pharmaceutically acceptable salt thereof after storage for 6 months, 9 months, or 1 year at about 25° C. and 60% relative humidity, or both. The oral dosage form more preferably contains at least about 90% w/w and even more preferably 95% w/w of undegraded bupropion or a pharmaceutically acceptable salt thereof and/or undegraded escitalopram or a pharmaceutically acceptable salt thereof after storage for 6 months, 9 months, or 1 year under the same conditions.

According to another embodiment, the oral dosage form provides modified release of the bupropion or pharmaceutically acceptable salt thereof. Preferably, the oral dosage form, upon ingestion by a patient, provides one or more of the following:
(a) a statistically significant lower mean fluctuation index for bupropion or a pharmaceutically acceptable salt thereof (preferably bupropion hydrochloride) in the plasma than an immediate release tablet containing the same amount of the bupropion or pharmaceutically acceptable salt thereof, and
(b) bioavailability of bupropion substantially equivalent to that of three immediate release tablets of the same form of bupropion administered one tablet every 6 or more hours, for one day (e.g., the AUC of the bupropion provided by the oral dosage form is 75% to 130% of that provided by the three immediate release tablets).

According to yet another embodiment, less than about 30% of the bupropion (or pharmaceutically acceptable salt thereof) (based on 100% bupropion in the oral dosage form) is released within 2 hours after administration, and more than about 60% of the bupropion is released 12 hours after administration.

According to yet another embodiment, the oral dosage form provides pulsated or sustained release, including delayed release and extended release, of the bupropion or pharmaceutically acceptable salt thereof. According to one embodiment, the oral dosage form, upon ingestion by a patient, releases the bupropion or pharmaceutically acceptable salt thereof in two or more pulses and preferably in three pulses. Each pulse is released at a different time after administration with time intervals between the release of the pulses during which substantially no bupropion or pharmaceutically acceptable salt thereof is released from the oral dosage form. Each pulse may be released under different conditions, e.g., at different times and/or at different pHs. For example, according to one embodiment, release of bupropion (or pharmaceutically acceptable salt thereof) is delayed by about 2 hours of ingestion, e.g., less than 20% is released and more than 60 % of bupropion is released in about 2 to about 12 hours after ingestion.

According to yet another embodiment, the oral dosage form includes modified release beads or tablets of bupropion (or a pharmaceutically acceptable salt thereof) to provide for its modified release. The beads and/or tablets may provide single phase or multi-phase release of the bupropion. According to one embodiment, the beads and/or tablets comprise a modified release bupropion core and one or more bupropion release layers. For example, the beads and/or tablets can have an immediate release bupropion layer and a modified release bupropion core. According to one embodiment, the tablets have a diameter ranging from about 4.5 to about 15 mm. The beads and/or tablets may be incorporated into a capsule.

According to one embodiment, the oral dosage form includes beads and/or tablets of bupropion (or a pharmaceutically acceptable salt thereof) having at least two different release profiles. For example, the oral dosage form may include buproprion immediate release beads and/or tablets and buproprion modified release beads and/or tablets.

According to yet another embodiment, the oral dosage form has an in vitro dissolution profile as measured by the USP Paddle Method at (a) 75 rpm in 900 mL of water at 37° C. or (b) 100 rpm in 900 mL 0.1 N HCl at 37° C., such that (i) after 2 hours, less than about 30% by weight of the bupropion (or a pharmaceutically acceptable salt thereof) is released, (ii) after 8 hours, from about 40% to about 90% is released, and (iii) after 24 hours, more than about 70% is released.

The aforementioned oral dosage forms may provide immediate or modified release of the escitalopram or pharmaceutically acceptable salt thereof. Dosage forms formulated for the immediate release of escitalopram oxalate preferably provide a Tₘₐₓ ranging from about 1 to about 8 hours, and more preferably about 5 hours. The modified release of escitalopram oxalate preferably provides a Tₘₐₓ of approximately 4 to 24 hours.

According to a preferred embodiment, the oral dosage form, upon ingestion by a subject, provides at least one of the following:
(a) a mean Cₘₐₓ for escitalopram (or pharmaceutically acceptable salt thereof) that is about 50 to about 85% of that for an immediate release tablet containing the same amount of the escitalopram (or pharmaceutically acceptable salt thereof),
(b) a Tₘₐₓ for escitalopram (or pharmaceutically acceptable salt thereof) of from about 4 to about 12 hours,
(c) bioavailability for escitalopram (or pharmaceutically acceptable salt thereof) substantially equivalent to that of an immediate release tablet containing the same amount of the escitalopram (or pharmaceutically acceptable salt thereof) (e.g., the AUC of the escitalopram provided by the oral dosage form is 75% to 130% of that provided by the immediate release tablet),
(d) a statistically significant lower mean fluctuation index (difference between Cmax and Cmin) in the plasma for escitalopram than an immediate release tablet containing the same amount of the escitalopram (or pharmaceutically acceptable salt thereof),
(e) a mean minimum plasma concentration (Cₘᵢₙ) for escitalopram (or pharmaceutically acceptable salt thereof) substantially equivalent to that of an immediate release tablet containing the same amount of the escitalopram (or pharmaceutically acceptable salt thereof), and
(f) an area under a plasma concentration vs. time curve (AUC) for escitalopram (or pharmaceutically acceptable salt thereof) within the range of from about −25% to about +30% of that produced by an immediate release tablet containing the same amount of the escitalopram (or pharmaceutically acceptable salt thereof). According to a more preferred embodiment, for example, 8 mg of the oral dosage form provides an AUC₀₋₂₄ for escitalopram oxalate of about 200 to about 350 ng·h/ml. The comparative immediate release tablet is preferably 4 and 8 mg and the 5, 10, or 20 mg escitalopram oxalate tablet which is the subject of United States Food and Drug Administration Approved New Drug Application No. 21-323.

The immediate release dosage form preferably has an in vitro dissolution profile for escitalopram (as measured by the USP Basket Method at 100 rpm in 900 ml 0.1 N HCl at 37° C.) such that more than 80% of the drug is released in about 30 minutes. The release dosage form preferably has an in vitro dissolution profile for escitalopram (as measured using by the USP Basket Method at 100 rpm in 900 ml 0.1 N HCl at 37° C.) such that within 2 hours, from about 10% to about 50% by weight of the escitalopram is released, and after 8 hours, more than about 70% by weight of the escitalopram is released.

According to a preferred embodiment, the oral dosage form e.g., comprising about 8 mg of escitalopram or a pharmaceutically acceptable salt thereof and, upon ingestion by a patient, provides a mean maximum plasma concentration (Cₘₐₓ) of the escitalopram from about 2 to about 25 ng/ml, and more desirably from about 3 to about 15 ng/ml.

According to yet another embodiment, the oral dosage form includes modified release beads or tablets of escitalopram (or a pharmaceutically acceptable salt thereof) to provide for its modified release. The beads and/or tablets may provide single phase or multi-phase release of the escitalopram. According to one embodiment, the beads and/or tablets comprise a modified release escitalopram core and one or more escitalopram release layers. For example, the beads and/or tablets can have an immediate release escitalopram layer and a modified release escitalopram core. According to one embodiment, the tablets have a diameter ranges from about 4.5 to about 15 mm. The beads and/or tablets may be incorporated into a capsule.

According to one embodiment, the oral dosage form includes beads and/or tablets of escitalopram (or a pharmaceutically acceptable salt thereof) having at least two different release profiles. For example, the oral dosage form may include escitalopram immediate release beads and/or tablets and escitalopram modified release beads and/or tablets.

Yet another embodiment is a method of treating a central nervous system (CNS) disorder (such as a mood or anxiety disorder) in a patient in need thereof by daily administration an oral dosage form of the present invention. Examples of CNS disorders which can be treated include, but are not limited to, major depressive disorder, general anxiety disorder, social anxiety disorder, post traumatic stress disorder, panic attacks, acute stress disorder, eating disorders (such as bulimia, anorexia and obesity), phobias, dysthymia, premenstrual syndrome, premenstrual dysphoric disorder, cognitive disorders, impulse control disorders, attention deficit hyperactivity disorder and drug abuse. The combination of escitalopram and bupropion can also effectively treat patients who have failed to respond to initial treatment with a conventional SSRI, in particular patients with major depression disorder who have failed to respond to initial treatment with a conventional SSRI. The combination can further treat or reduce suicidal thoughts in a patient in need thereof, and improve disability free survival following stroke.

Yet another embodiment is a method of treating a patient suffering from treatment resistant depression by administering the oral dosage form of the present invention.

Another embodiment of the invention is a method of treating a patient suffering from nausea, insomnia, somnolence, increased sweating, fatigue, or a combination thereof due to treatment with an antidepressant other than a combination of bupropion or a pharmaceutically acceptable salt thereof and escitalopram or a pharmaceutically acceptable salt thereof. The method includes (a) discontinuing treatment with the antidepressant; and (b) treating the patient with an oral dosage form of the present invention. According to one embodiment, the antidepressant is an immediate release escitalopram oxalate formulation.

Yet another embodiment is a method for treating sexual dysfunction in a patient suffering from sexual dysfunction due to treatment with an antidepressant other than a combination of escitalopram or a pharmaceutically acceptable salt thereof and bupropion or a pharmaceutically acceptable salt thereof. The sexual dysfunction may be ejaculation disorder, anorgasmia, and/or decreased libido. The method includes (a) discontinuing treatment with the antidepressant; and (b) treating the patient with an oral dosage form of the present invention. According to one embodiment, the oral dosage form provides release of bupropion such that a first release of bupropion (or pharmaceutically acceptable salt thereof) is delayed by about 2 hours of ingestion, e.g., less than 20% is released and more than 60% of bupropion is released about 2 to about 12 hours Optionally, additional pulses can be released such that bupropion hydrochloride is released (>80%) approximately 4 to 24 hours after ingestion.

### BRIEF DESCRIPTION OF THE DRAWINGS

FIG. 1 shows the simulated dissolution profile of escitalopram core beads (200 mg/g) and, modified release beads (194.1 mg/g and 188.7 mg/g) described in Example 1.

FIG. 2 shows the simulated dissolution profile of bupropion core beads (600 mg/g) and modified release beads (545.5 mg/g and 500 mg/g) described in Example 3.

FIG. 3 shows the bupropion dissolution profile of the three capsules described in Table 14 of Example 5 as determined by the USP basket method at 100 rpm in 0.1 N HCl.

FIG. 4 shows the pulsatile escitalopram beads dissolution profile of the prepared by (1) mixing immediate release and modified release beads and (2) unitary beads comprising of immediate and modified release described in Table 14 of Example 5 as determined by the USP basket method at 100 rpm in 0.1 N HCl.

### DETAILED DESCRIPTION OF THE INVENTION

Definitions

The term “escitalopram” as used herein includes 1-[3-(dimethyl-amino)propyl]-1-(p-fluorophenyl)-5-phthalancarbonitrile preferably containing less than 3, 2, 1, 0.5, or 0.2% by weight of its R-enantiomer (based on 100% total weight of 1-[3-(dimethyl-amino)propyl]-1-(p-fluorophenyl)-5-phthalancarbonitrile), i.e., S-citalopram having an enantiomeric purity (by weight) of 97, 98, 99, 99.5, or 99.8%. Pharmaceutically acceptable salts of escitalopram include, but are not limited to, acid addition salts formed with organic and inorganic acids. Non-limiting examples of suitable organic acids are maleic, fumaric, benzoic, ascorbic, pamoic, succinic, oxalic, salicylic, methanesulfonic, ethanedisulfonic, acetic, propionic, tartaric, citric, gluconic, lactic, malic, mandelic, cinnamic, citraconic, aspartic, stearic, palmitic, itaconic, glycolic, p-amino-benzoic, glutamic, benzene sulfonic and theophylline acetic acid, as well as the 8-halotheophyllines, for example, 8-bromotheophylline. Non-limiting examples of suitable inorganic acids are hydrochloric, hydrobromic, sulfuric, sulfamic, phosphoric and nitric acids. Preferred pharmaceutically acceptable salts of escitalopram include, but are not limited to, escitalopram oxalate and escitalopram hydrobromide. The term “escitalopram” also includes polymorphs, hydrates, solvates, and amorphous forms of escitalopram and its pharmaceutically acceptable salts. Escitalopram and pharmaceutically acceptable salts thereof can be prepared as described in U.S. Pat. Nos. Re. 34,712 and 6,566,540 and International Publication Nos. WO 03/000672, WO 03/006449, WO 03/051861, and WO 04/083197, the disclosures of which are each hereby incorporated by reference in their entirety. Crystals of escitalopram oxalate and escitalopram hydrobromide such as those described in International Publication No. WO 03/011278, U.S. Patent Application Publication No. 2004/0167209, and U.S. patent application Ser. Nos. 10/851,763 and 10/948,594, all of which are hereby incorporated by reference, can also be used. The comparative escitalopram “immediate release” tablets referred to herein are preferably those of United States Food and Drug Administration Approved New Drug Application No. 21-323 of equal amount (5, 10 and 20 mg escitalopram as oxalate).

Unless specified otherwise, all weight values of escitalopram salts are provided as the weight equivalent of escitalopram free base. For example, 4 mg escitalopram oxalate refers to an amount of escitalopram oxalate which is a molar equivalent to 4 mg escitalopram free base.

The term “bupropion” refers to (±)-1-(3-chlorophenyl)-2-[(1,1-dimethylethyl)amino]-1-propanone. Pharmaceutically acceptable salts of bupropion include, but are not limited to, acid addition salts formed with organic or inorganic acids, for example, hydrochloride, hydrobromide, sulphate, nitrate, phosphate, formate, mesylate, citrate, benzoate, fumarate, maleate and succinate. The term “bupropion” also includes polymorphs, hydrates, solvates, and amorphous forms of bupropion and its pharmaceutically acceptable salts. A preferred pharmaceutically acceptable salt of bupropion is bupropion hydrochloride. The comparative bupropion “immediate release” tablets referred to herein are preferably those of New Drug Application No. 018-644 of equal amount (50, 75, and 100 mg bupropion hydrochloride).

An “effective amount” means the amount of an active ingredient or a combination of active ingredients that, when administered to a mammal for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated. According to one embodiment of the present invention, an effective amount of escitalopram is an amount effective to treat a central nervous system (CNS) disorder, such as, major depressive disorder, general anxiety disorder, social anxiety disorder, post traumatic stress disorder, or panic attacks.

The term “pharmaceutically acceptable” generally means biologically or pharmacologically compatible for in vivo use in animals or humans, and preferably means approved by a regulatory agency of the Federal or a state government or listed in the U.S. Pharnacopeia or other generally recognized pharmacopeia for use in animals, and more particularly in humans.

As used herein, the term “treat” includes one or more of the following:

(a) relieving or alleviating at least one symptom of a disorder in a subject, including for example, central nervous system (CNS) disorders, (such as, mood disorders, major depressive disorder, general anxiety disorder, social anxiety disorder, post traumatic stress disorder, and panic attacks, including panic attacks);

(b) relieving or alleviating the intensity and/or duration of a manifestation of a disorder experienced by a subject including, but not limited to, those which are in response to a given stimulus (e.g., pressure, tissue injury, and cold temperature); and

(c) arresting, delaying the onset (i.e., the period prior to clinical manifestation of a disorder) and/or reducing the risk of developing or worsening a disorder.

The term “panic attacks” includes, but is not limited to, any disease, which is associated with panic attacks including panic disorder, specific phobias, social phobia and agoraphobia in which panic attacks occur. These disorders are further defined in the Diagnostic and Statistical Manual of Mental Disorders 4^{th} Ed.—Text Revision (DSM-IV-TR), A. Frances (ed.), American Psychiatric Association, Washington, D.C., 2000). A panic attack is a discrete period in which there is a sudden onset of intense apprehension, fearfulness or terror, often associated with feelings of impending doom. During the attack, symptoms such as palpitations, sweating, trembling, sensations of shortness of breath, feeling of choking, chest pain or discomfort, nausea, feeling dizzy, feelings of unreality, fear of losing control or going crazy, fear of dying, paresthesias and chills or hot flushes are present.

Panic disorders are characterized by recurrent unexpected panic attacks about which there is a persistent concern. Agoraphobia is anxiety about, or avoidance of, places or situations from which escape might be difficult or in which help may not be available in the event of a panic attack. Specific phobia and social phobia (together formerly simple phobia) are characterized by marked and persistent fear that is excessive or unreasonable, cued by the presence or anticipation of a specific object or situation (flying, heights, animals, seeing blood etc.) or social performance situations.

The disorders in which panic attacks occur are differentiated from each other by the predictability of the occurrence of the attacks, for example, in panic disorder the attacks are unpredictable and not associated with any particular event, whereas in specific phobia the attacks are triggered by specific stimuli.

The phrase “treatment of panic disorder” can include a reduction in the number or prevention of panic attacks and/or relief of the severity of the panic attacks.

The term “mood disorder” as used herein includes the mood disorders specified in the DSM-IV-TR, including, but not limited to, depressive disorders, such as major depressive disorder.

The term “anxiety disorder” as used herein includes the anxiety disorders specified in the DSM-IV-TR, including, but not limited to, panic disorder without agoraphobia, panic disorder with agoraphobia, social phobia (previously known as social anxiety disorder), obsessive-compulsive disorder, posttraumatic stress disorder, and generalized anxiety disorder.

Patients suffering from “treatment resistant depression” include (1) those who fail to respond to standard doses (i.e., significantly superior to placebo in double-blind studies) of antidepressants (such as SSRIs) administered continuously for a minimum duration of 6 weeks, and (2) those who fail to respond to standard doses of an antidepressant (such as an SSRI) (monotherapy) administered continuously for a minimum duration of 12 weeks. One criteria for determining whether a patient's depression is treatment resistant to an antidepressant is if a Clinical Global Impression-Improvement (CGI-I) score of 1 (very much improved) or 2 (much improved) is not achieved by the end of a 6, 8, or 12 week trial. The CGI-I scale is defined in Guy, W. (ed.): ECDEU Assessment Manual for Psychopharmacology, Revised, DHEW Pub. No. (ADM) 76-338, Rockville, Md., National Institute of Mental Health, 1976.

The terms “sustained release”, “modified release”, and “sustained or modified release” as used herein refer to the release of an active ingredient over an extended period of time leading to lower peak plasma concentrations and a prolonged Tₘₐₓ as compared to immediate release formulations. These terms also include release over a period of time via a series of immediate release pulses. The pharmacokinetic profile for 100 mg Wellbutrin® tablets (immediate release bupropion hydrochloride tablets) shows a peak plasma concentration at approximately 1-2 hours following administration. The pharmacokinetic profile for 20 mg escitalopram oxalate tablets (immediate release tablets) show a peak plasma concentration at approximately 5 hours. (Physician's Desk Reference 2005, Thomson Healthcare; 59th ed. 2004).

By “pulsatile” is meant that a plurality of drug doses are released at spaced apart time intervals.

The term “bioavailability” refers to the rate and extent to which the active ingredient or active moiety, e.g., escitalopram, is absorbed from a drug product and becomes systematically available.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean a range of up to 10%, preferably up to 5%.

Combinations of Bupropion and Escitalopram

The oral dosage form preferably includes about 75, 150, or 225 mg of bupropion hydrochloride and about 2.5, 4, 5, 10, 15, or 20 mg of escitalopram or a pharmaceutically acceptable salt thereof (such as escitalopram oxalate or escitalopram hydrobromide). In another embodiment, the oral dosage form preferably includes about 75, 150, or 300 mg of bupropion hydrochloride and about 4, 8, 12, 16, or 24 mg of escitalopram or a pharmaceutically acceptable salt thereof.

More preferred amounts of each component in the oral dosage form include, but are not limited to, those shown in the table below.

| | AMOUNT OF | AMOUNT OF |
|---|---|---|
| | ESCITALOPRAM (OR | BUPROPION (OR |
| | PHARMACEUTICALLY | PHARMACEUTICALLY |
| | ACCEPTABLE SALT | ACCEPTABLE SALT |
| NO. | THEREOF) | THEREOF) |
| 1 | 4 MG | 150 MG |
| 2 | 16 MG | 300 MG |

Unitary dosage forms containing both escitalopram and bupropion are preferably formulated so that the escitalopram and bupropion are not in contact with one another.

Modified Release Formulations

Preferably, the oral dosage forms containing the bupropion and/or escitalopram may be formulated to provide modified release of the bupropion and immediate and/or modified release of the escitalopram. The modified release profiles for bupropion, escitalopram, or both can be achieved by sustained, including delayed and extended release, and pulsatile formulations.

Pulsatile Formulations

Pulsatile release profiles can be achieved with dosage forms that are closed, such as sealed capsules or tablets, which contain two or more drug-containing dosage units. The dosage form can include one, two, three, or four or more types of dosage units, each having a different drug release profile. Each dosage unit can provide multi-phase release of the bupropion and/or escitalopram.

Preferably, the dosage form includes at least two types of dosage units, and more preferably, includes two or three types of dosage units. For example, according to one embodiment, the first type of dosage unit releases drug substantially immediately following ingestion of the dosage form, the second type releases drug approximately 1 to 8 hours following ingestion, and the optional third type releases drug approximately 2 to 24 hours following ingestion.

According to another embodiment, about 20 to 60% of the escitalopram and about 10 to 50% of the bupriopion is released in a first pulse. The release of the bupropion and any remaining escitalopram occurs in one or more pulses following the first pulse. The number of pulses and amount of the drugs released preferably result in a Tₘₐₓ of from about 4 to about 24 hours for escitalopram and from about 4 to about 12 hours for bupropion.

Each dosage unit can be, for example, a tablet (e.g., compressed or molded), bead, or particle. Alternatively, the dosage units may be different layers on the dosage form (e.g., a multi-layered tablet). Suitable pulsatile systems are described in U.S. Pat. Nos. 6,217,904, 6,555,136, 6,793,936, 6,627,223, 6,372,254, 6,730,321, 6,500,457, 4,723,958, 5,840,329, 5,508,040, and 5,472,708 and U.S. Patent Application Publication Nos. 2003-124196, 2004-028729, and 2003-0133978, the disclosures of which are hereby incorporated by reference in their entirety.

The tablet dosage units can be of any size. According to one preferred embodiment, the tablets have a major diameter axis ranging from about 4.5 to about 15 mm. According to one embodiment, the dosage form (e.g., a capsule) contains two or three tablets.

Generally, the bead dosage units comprise an inert support with a drug coated thereon and/or a drug containing core. The inert support can be, for example, a bead of sugar or microcrystalline cellulose. The drug can be coated on the inert support by methods known in the art.

The individual dosage units (such as beads and particles) can be compacted or compressed into a single tablet or capsule by methods known in the art.

Sustained Release Formulations

Sustained release profiles for a dosage form can be achieved by coatings and/or the use of the aforementioned beads, particles, and tablets as dosage units within the dosage form.

Dosage Units

As will be appreciated by those skilled in the art and as described in the pertinent texts and literature, a number of methods are available for preparing drug-containing tablets or other dosage units which provide a variety of drug release profiles. Such methods include coating a drug or drug-containing composition, increasing the drug's particle size, placing the drug within a matrix, and forming complexes of the drug with a suitable complexing agent.

The modified release dosage units for the pulsatile and sustained release formulations can be prepared, for example, by coating a drug or a drug-containing composition with one or more membrane coating materials, such as one or more polymeric materials. When a coating is used to provide delayed release dosage units, particularly preferred coating materials include, but are not limited to, bioerodible, gradually hydrolyzable and/or pH dependent soluble polymers. The “coating weight,” or relative amount of coating material per dosage unit, and the type of polymer generally dictates the time interval between ingestion and drug release.

Suitable membrane coating materials for effecting delayed release include, but are not limited to: cellulosic polymers such as cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose hexahydrophthalate, cellulose acetate hexahydrophthalate, and carboxymethylcellulose sodium; acrylic acid polymers and copolymers preferably formed from acrylic acid,. methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate (such as a terpolymer of ethyl acrylate, methyl methacrylate and trimethylammonioethyl methacrylate chloride (available as Eudragit® RS from Röhm America L.L.C., of Piscataway, N.J.)); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac, ammoniated shellac, shellac-acetyl alcohol, and shellac n-butyl stearate.

In some cases, it may be desirable for a tablet, bead, or particle to provide for release of the drug in the colon, in which case polymeric or other materials are used that enable drug release within the colon. These may be selected from the aforementioned list, or other materials may be used as will be known to those skilled in the art of pharmaceutical formulation and drug delivery. For example, hydrocolloid gums may be effective to provide for colonic delivery, e.g., guar gum, locust gum, bena gum, gum tragacanth, and karaya gum (see, e.g., U.S. Pat. No. 5,656,294). Other materials suitable for effecting colonic drug delivery include polysaccharides, mucopolysaccharides, and related compounds, e.g., pectin, arabinogalactose, chitosan, chondroitin sulfate, dextran, galactomannan, and xylan.

The desired pulsatile profile may be achieved by a dosage form comprised of a plurality of tablets. The first tablet is provided with little or no coating material, the second tablet is provided with some degree of coating material, the third tablet is provided with even more coating material, and so on. Analogously, for encapsulated dosage forms in which the drug-containing dosage units are beads or particles, a first fraction of beads or particles is provided with little or no functional coating material, a second fraction is provided with some degree of sustained release coating material, the third faction is provided with even more coating material, and so on. For example, when the dosage form contains three tablets (or, analogously, three types of drug-containing particles or beads), the first tablet, which releases drug substantially immediately, may have a total coating weight of less than about 5% (preferably less than about 3%) (based on the total weight of the tablet), the second tablet may have a total coating weight in the range of approximately 5% to 50% (preferably 5% to 40%) and the third tablet, if present, may have a total coating weight in the range of approximately 25% to 60% (preferably 25% to 50%). The preferred coating weights for particular coating materials may be readily determined by those skilled in the art by evaluating individual release profiles for dosage units prepared with different quantities of various coating materials.

Alternatively, the delayed release dosage units, e.g., tablets, beads, or particles, may be formulated by using a polymer coating that imparts delayed release properties. The insoluble plastic matrices may be comprised of, for example, polyvinyl chloride or polyethylene. Hydrophilic polymers useful for providing a matrix for a delayed release dosage unit include, but are not limited to, those described above as suitable coating materials. The mixture of particles can be compressed into tablets or processed into individual drug-containing particles.

The individual dosage units may be provided with colored coatings, with a single color used to identify a tablet or bead or particle fraction having a corresponding delayed release profile. That is, for example, a blue coating may be used for the immediate release tablet or bead or particle fraction, a red coating may be used for the “medium” release tablet or bead or particle fraction, and so on. In this way, errors during manufacture can be easily avoided. The color is introduced by incorporating a pharmaceutically acceptable colorant into the coating during coating preparation. The colorant may be either natural or synthetic. Natural colorants include, but are not limited to, pigments such as chlorophyll, anattenes, beta-carotene, alizarin, indigo, rutin, hesperidin, quercitin, carminic acid, and 6,6′-dibromoindigo. Synthetic colorants include, but are not limited to, dyes, including both acidic dyes and basic dyes, such as nitroso dyes, nitro dyes, azo dyes, oxazines, thiazines, pyrazolones, xanthenes, indigoids, anthraquinones, acridines, rosanilines, phthaleins, and quinolines.

For encapsulated tablets, the weight of each individual tablet in the capsule is typically in the range of about 50 mg to about 750 mg, preferably in the range of about 50 mg to about 600 mg, and more preferably in the range of about 60 mg to about 450 mg. The individual tablets can be prepared by methods known in the art. A preferred method for forming tablets herein is by direct compression of a powdered, crystalline or granular drug-containing composition, alone or in combination with diluents, binders, lubricants, disintegrants, colorants or the other excipients. Compressed tablets can also be prepared by wet-granulation or dry-granulation processes. Tablets may also be molded rather than compressed, starting with a moist material containing a suitable water-soluble lubricant. Drug-containing particles or beads may also be prepared by methods known in the art, such as with a fluid dispersion.

Coating procedures and equipment known in the art may be used to coat the dosage units, e.g., the drug-containing tablets, beads or particles. For example, a delayed release coating composition may be applied using a coating pan, or fluidized bed coating equipment. Materials, equipment and processes for preparing tablets, beads, drug particles, and delayed release dosage forms are described in Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6^{th} Ed. (Media, Pa.: Williams & Wilkins, 1995).

Optional components present in the individual drug-containing dosage units include, but are not limited to, diluents, binders, lubricants, disintegrants, surfactants, and coloring agents.

Diluents (also referred to as “fillers”) are typically included to increase the bulk of a tablet so that a practical size is provided for compression. Suitable diluents include, but are not limited to, dicalcium phosphate dihydrate, calcium sulfate, lactose, cellulose, kaolin, mannitol, sodium chloride, dry starch, hydrolyzed starches, silicon dioxide, titanium oxide, alumina, talc, microcrystalline cellulose, powdered sugar, and mixtures thereof.

Binders are used to impart cohesive qualities to a tablet formulation, and thus ensure that a tablet remains intact after compression. Suitable binders include, but are not limited to, starch (including corn starch and pregelatinized starch), gelatin, sugars (including sucrose, glucose, dextrose, lactose and sorbitol), polyethylene glycol, waxes, natural and synthetic gums (e.g., acacia, tragacanth, sodium alginate, polyvinylpyrrolidone, celluloses, and Veegum), and synthetic polymers (such as polymethacrylates and polyvinylpyrrolidone), and mixtures thereof.

Lubricants are used to facilitate tablet manufacture. Examples of suitable lubricants include, but are not limited to, magnesium stearate, calcium stearate, stearic acid, glyceryl behenate, and polyethylene glycol. Preferably, a dosage unit contains no more than approximately 1 wt. % (relative to the weight of the dosage unit) of lubricant.

Disintegrants are used to facilitate tablet disintegration or “breakup” after administration. Suitable disintegrants include, but are not limited to, starches, clays, celluloses, algins, gums, crosslinked polymers, and mixtures thereof.

Stabilizers are used to inhibit or retard drug decomposition reactions which include, by way of example, hydrolysis, such as those involving bupropion and pharmaceutically acceptable salts thereof (e.g., bupropion hydrochloride). Suitable. stabilizers includes those described in U.S. Pat. Nos. 5,763,493, 5,731,000, and 5,358,970. The stabilizer can be an organic acid, a carboxylic acid, an acid salt of an amino acid, sodium metabisulphite, or a mixture thereof. Examples of acid salts of amino acids include, but are not limited to, hydrochloride salts such as cysteine hydrochloride, L-cysteine hydrochloride, glycine hydrochloride, and cystine dihydrochloride. Examples of other stabilizers include, but are not limited to, ascorbic acid, malic acid, isoascorbic acid, citric acid, and tartaric acid.

According to one embodiment, oral dosage forms of bupropion may be prepared using non-organic solvents. According to another embodiment of the present invention, the dosage form contains less than 0.2% BHT, anti-oxidant.

Suitable surfactants include, but are not limited to, anionic, cationic, amphoteric, and nonionic surface active agents. Suitable anionic surfactants include, but are not limited to, those containing carboxylate, sulfonate and sulfate ions, associated with cations such as sodium, potassium and ammonium ions. Other suitable surfactants include, but are not limited to, long alkyl chain sulfonates and alkyl aryl sulfonates, such as sodium dodecylbenzene sulfonate; dialkyl sodium sulfosuccinates, such as sodium bis-(2-ethylhexyl)-sulfosuccinate; and alkyl sulfates such as sodium lauryl sulfate.

If desired, the tablets may also contain non-toxic auxiliary substances such as wetting or emulsifying agents, pH buffering agents, and preservatives.

As noted earlier herein, the individual drug tablets, beads or particles are, in one embodiment, contained within a closed capsule. The capsule material may be either hard or soft, and as will be appreciated by those skilled in the art of pharmaceutical science, typically comprises a tasteless, easily administered and water soluble compound such as gelatin, starch or cellulose. A preferred capsule material is gelatin. The capsules are preferably sealed, such as with gelatin bands. See, for example, Remington: The Science and Practice of Pharmacy, 20^{th} Edition (Easton, Pa.: Mack Publishing Co., 2000), which describes materials and methods for preparing encapsulated pharmaceuticals designed to dissolve shortly after ingestion.

Dosage Forms

The dosage form may also include one or more release modifiers in the form of polymeric coatings or matrices. The dosage form can also include one or more carriers, excipients, anti-adherants, fillers, stabilizing agents, binders, colorants, glidants, and lubricants.

Depending upon the hydrophilic or hydrophobic nature of the matrix, it may be a material that swells upon contact with gastric fluid to a size that is large enough to promote retention in the stomach while the subject is in the digestive state. The digestive state is induced by food ingestion and begins with a rapid and profound change in the motor pattern of the upper gastrointestinal (GI) tract. The change consists of a reduction in the amplitude of the contractions that the stomach undergoes and a reduction in the pyloric opening to a partially closed state. The result is a sieving process that allows liquids and small particles to pass through the partially open pylorus while indigestible particles that are larger than the pylorus are retropelled and retained in the stomach. Biological fluids migrate through the matrix and dissolve the active ingredient which is released by diffusion through the matrix, which simultaneously modulates the release flow. The controlled-release matrix in these embodiments of the invention is therefore selected as one that can swell to a size large enough to be retropelled and thereby retained in the stomach, causing the prolonged release of the drug to occur in the stomach rather than in the intestines. Disclosures of oral dosage forms that swell to sizes that will prolong the residence time in the stomach are found in U.S. Pat. Nos. 5,007,790, 5,582,837, and 5,972,389, as well as International Publication Nos. WO 98/55107 and WO 96/26718. Each of the documents cited in this paragraph is incorporated herein by reference in its entirety.

The matrix may be composed of an insoluble hydrophilic polymer, such as a cellulose ester, carboxyvinyl ester, or acrylic or methacrylic ester. On contact with biological fluids, the hydrophilic matrix becomes hydrated and swells, forming a very dense network of polymers, through which the soluble active principles diffuse. Furthermore, lipids, in particular glyceryl esters, can be added in order to modulate the matrix swelling. These compositions can be obtained by granulation and then compression of the mixture formed of the polymer, active principles and various adjuvants.

Hydrophobic matrices can be composed of a lipid matrix agent of natural origin, for example beeswaxes, which is highly innocuous. These compositions can be obtained by granulation, by a wet or solvent route, and then compression involving high proportions of each of the constituents.

In general, swellable matrices contain binders that are water-swellable non-toxic polymers, swell in a dimensionally unrestricted manner upon imbibitions of water, and release the drug gradually over time. Examples of polymers meeting this description include, but are not limited to the following: cellulose polymers and their derivatives including, but not limited to, hydroxymethyl cellulose, hydroxyethyl cellulose, hydroxypropyl cellulose, hydroxypropylmethyl cellulose, carboxymethylcellulose, and microcrystalline cellulose polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, maltodextrins, poly (2-ethyl-2-oxazoline), poly(ethyleneimine), polyurethane hydrogels, and crosslinked polyacrylic acids and their derivatives. Further non-limiting examples are copolymers of the polymers listed above, including block copolymers and graft polymers. Specific examples of copolymers are PLURONIC® and TECTONIC®, which are polyethylene oxide-polypropylene oxide block copolymers and available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Other suitable polymers for the matrices are poly(ethylene oxide), hydroxypropyl methyl cellulose, and combinations of poly(ethylene oxide) and hydroxypropyl methyl cellulose. A preferred polymer is hydroxypropyl methyl cellulose. In one embodiment, modified release formulations, such as a 24-hour modified release formulation, contain such polymers in amounts ranging from about 10% w/w to about 50% w/w, and preferably from about 15% to about 45% w/w.

The prolongation in the time of maximum plasma concentration values (Tₘₐₓ) as compared to immediate release, is related to the in vitro dissolution release rate of the drug. The in vitro dissolution release rate of the drug depends on the composition of the matrix. By using different cellulosic matrices, in-vitro release rates (drug dissolution of more than about 70% to about 80%) can be manipulated anywhere from about 4 hours to 24 hours. The formulations have a time of maximum plasma concentration (average Tₘₐₓ) ranging from between about 1 to about 35 hours for both drugs, preferably from about 4 to about 30 hours and an in vitro release rate of more than about 70% to about 80% in about 4 to about 24 hours. Preferably, the formulations have a release rate for escitalopram from about more than 80% in about 30 minutes to about 12 hours. More preferably, the formulations have a release rate of about 10% to about 40% within the first hour following entry into a use environment (such as the gastrointestinal tract) followed by extended release; and more preferably, the formulations have a release rate of more than 70% within the next 12 hours.

Tablets in accordance with this invention can be prepared by conventional mixing, comminution, and tabletting techniques that are well known in the pharmaceutical formulations industry. The modified-release tablet, for example, may be fabricated by direct compression by punches and dies fitted to a rotary tabletting press, ejection or compression molding, granulation followed by compression, or forming a paste and extruding the paste into a mold or cutting the extrudate into short lengths.

Fillers such as lactose (e.g., lactose monohydrate) are used to modify the dissolution pattern. When hydroxypropyl methylcellulose or ethyl cellulose are used, the dissolution rates can be much slower than the modified release rate targeted. The slow release is because hydrophobic matrix tablets that are formed release the drug by the mechanism of polymer erosion. Since the erosion from a hydrophobic matrix is very slow, the dissolution rate of the readily soluble active ingredient is also slow. Lactose, however, is also an important filler ingredient useful in improving the powder flow and compressibility for escitalopram and bupropion tablets.

When tablets are made by direct compression, the addition of lubricants may be helpful and is sometimes important to promote powder flow and to prevent capping of the tablet (the breaking off of a portion of the tablet) when the pressure is relieved. Useful lubricants include magnesium stearate, and hydrogenated vegetable oil (preferably hydrogenated and refined triglycerides of stearic and palmitic acids). In a preferred embodiment, the lubricant is magnesium stearate. For 24-hour release formulations, the magnesium stearate preferably is present in amounts ranging from about 0.5% w/w to about 3% w/w, and preferably from about 0.5% w/w to about 2% w/w. Additional excipients may be added to enhance tablet hardness, powder flowability, and tablet friability and to reduce adherence to the die wall.

### EXAMPLE 1

### Escitalopram Core and Modified Release Beads

Tables 1 and 2 show the formulation ingredients and weight percent ranges for the manufacture of escitalopram core and modified release beads, respectively. Each modified release bead is an escitalopram core bead coated with a modified release coating.

TABLE 1
Escitalopram Core Bead Formulation Ranges

| Ingredients | % w/w |
|---|---|
| Escitalopram Oxalate | 3.0-70.0 |
| Compritol ® 888* | 5.0-50.0 |
| Sorbitan Monostearate | 1.0-15.0 |
| Avicel ® PH 101** | 3.0-50.0 |
| PVP K-30*** | 1.0-7.0 |
| Talc, USP | 1.0-6.0 |
| Sorbitan Oleate | 3.0-15.0 |
| Total | 100.0 |

*Compritol ® 888 is glyceryl behenate (tribehenin) and is available from Gattefosse Corp. of Paramus, N.J.
**Avicel ® PH 101 is microcrystalline cellulose and is available from FMC Corporation of Philadelphia, PA.
***PVP K-30 is polyvinylpyrrolidone having a K-value of about 30.

TABLE 2
Escitalopram Modified Release Coating Formulation Ranges

| Ingredients | % w/w |
|---|---|
| Escitalopram Core Beads | 20-95 |
| Surelease ®**** | 2-40.0 |
| Purified Water | QS |
| Total | 100.0 |

****Surelease ® is an aqueous ethylcellulose dispersion and is available from Colorcon, Inc. of West Point, PA.

Escitalopram core beads (200 mg/g) having the formulation in Table 3have been manufactured.

TABLE 3
Escitalopram Core Beads (200 mg/g)

| # | Ingredients | % w/w | Wt. in gms |
|---|---|---|---|
| 1. | Escitalopram Oxalate | 20.0 | 200 |
| 2. | Compritor ® 888 | 37.0 | 370 |
| 3. | Avicel ® PH 101 | 22.0 | 220 |
| 4. | PVP K-30 | 7.0 | 70 |
| 5. | Sorbitan Oleate | 10.0 | 100 |
| 6. | Talc, USP | 4.0 | 40 |
| | Total | 100.0 | 1000 |

The beads may be prepared by mixing ingredients 1-5 from Table 3 in a high shear granulator (Disona, Fluid Air, Chicago, Ill.). The granulated material is extruded with an extruder (Niro, Model E-140, Columbia Md.), and then spheronized into beads using a spheronizer (Niro Model S450, Columbia, Md.). The beads are optionally dried at 50° C. for up to 12 hours.

The escitalopram core beads from Table 3 have been coated with a modified release coating according to Table 4 (Profile I) or 5 (Profile II).

TABLE 4
Escitalopram Modified Release Beads (194.1 mg/g)

| Ingredients | % w/w |
|---|---|
| Escitalopram Core Bead, 200 mg /g | 97 |
| Surelease ® Dispersion (solid content) | 3 |
| Purified Water | QS |
| Total | 100.0 |

TABLE 5
Escitalopram Modified Release Beads (188.7 mg/g)

| Ingredients | % w/w |
|---|---|
| Escitalopram Core Bead, 200 mg/g | 94 |
| Surelease ® Dispersion (solid content) | 6 |
| Purified Water | QS |
| Total | 100.0 |

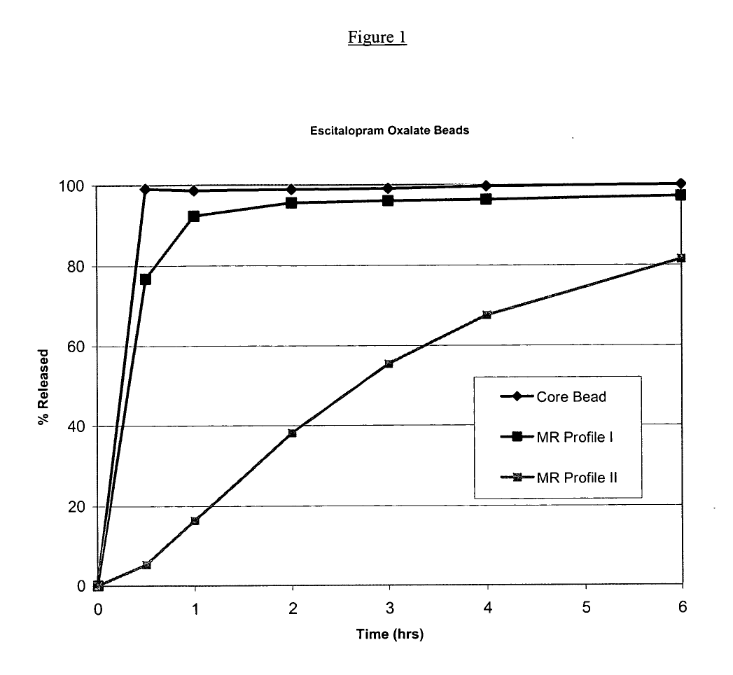

The simulated dissolution profiles of the escitalopram core beads (200 mg/g) of Table 3 and the modified release beads of Tables 4 and 5 are shown in FIG. 1.

### EXAMPLE 2

### Pulsatile Escitalopram Capsule Dosage Form

The escitalopram core and modified release beads described in Example 1 can be filled into capsules to deliver pulsatile release profiles. For example, predetermined weights of beads can be filled in a capsule using a capsule filling machine (MG-2, MG America, Fairfield, N.J.). The amounts of beads per capsule for a 4 mg strength pulsatile escitalopram capsule are shown in Table 6.

TABLE 6
Pulsative Escitalopram Capsule Dosage Formulations

| | | Modified | Modified |
|---|---|---|---|
| | Core Bead | release bead 1 | release Bead 2 |
| Profile | (mg/cap) | (mg/cap) | (mg/cap) |
| Single Pulse | 0 | 0 | 27.1 mg |
| Two Pulses | 6.4 mg | 0 | 20.3 mg |
| Three Pulses | 6.4 mg | 6.6 mg | 13.6 mg |

Capsules containing different amounts of beads of a given strength will generate different dissolution profiles. Also, different dose proportional strengths can be generated by using more beads, such for 5, 8, 10, 15, 16, 20, and 40 mg by the total fill weight.

### EXAMPLE 3

### Bupropion Core and Modified Release Beads

Tables 7 and 8 show the formulation ingredients and weight percent ranges for the manufacture of bupropion core and modified release beads, respectively. Each modified release bead includes a bupropion core bead coated with a modified release coating. Table 9 shows the formulation ingredients and weight percent ranges for Bupropion Core Beads (600 mg/g).

TABLE 7
Bupropion Core Bead Formulation Ranges

| INGREDIENTS | % W/W |
|---|---|
| BUPROPION HCL | 3.0-70.0 |
| COMPRITOL ® 888 | 5.0-50.0 |
| SORBITAN MONOSTEARATE | 1.0-15.0 |
| AVICEL ® PH 101 | 3.0-50.0 |
| HPMC | 0-30.0 |
| PVP K-30 | 1.0-7.0 |
| TALC, USP | 1.0-6.0 |
| SORBITAN OLEATE | 3.0-15.0 |
| TOTAL | 100.0 |

TABLE 8
Bupropion Modified Release Coating Formulation Ranges

| INGREDIENTS | % W/W |
|---|---|
| BUPROPION HCL CORE BEADS | 20-95 |
| EUDRAGIT ® | 5-40.0 |
| PURIFIED WATER | QS |
| TOTAL | 100.0 |

TABLE 9
Bupropion Core Beads (600 mg/g)

| # | INGREDIENTS | % W/W | WT. IN GMS |
|---|---|---|---|
| 1. | BUPROPION HCL | 60.0 | 600 |
| 2. | COMPRITOL ® 888 | 17.0 | 170 |
| 3. | AVICEL ® PH 101 | 7.0 | 70 |
| 4. | PVP K-30 | 2.0 | 20 |
| 5. | TALC, USP | 4.0 | 40 |
| 6. | SORBITAN OLEATE | 10.0 | 100 |
| | TOTAL | 100.0 | 1000 |

The beads may be prepared by mixing ingredients 1-5 from Table 9 in a high shear granulator (Disona, Fluid Air, Chicago, Ill.). The granulated material is extruded with an extruder (Niro, Model E-140, Columbia Md.), and then spheronized into beads using a spheronizer (Niro Model S450, Columbia, Md.). The beads are optionally dried at 50° C. for up to 12 hours.

The bupropion core beads from Table 9 have been coated with a modified release coating according to Table 10 (Profile I) or Table 11 (Profile II).

TABLE 10
Bupropion Modified Release Beads (545.5 mg/g)

| INGREDIENTS | % W/W |
|---|---|
| BUPROPION CORE BEAD 600 MG/G | 90 |
| EUDRAGIT ® RS/RL (95%:5%)* | 10 |
| PURIFIED WATER | QS |
| TOTAL | 100.00 |

*Eudragit ® RS/RL (95%:5%) is a mixture containing 95% Eudragit ® RS and 5% Eudragit ® RL, both of which are available from Röhm America Inc. of Piscataway, N.J.

TABLE 11
Bupropion Modified Release Beads (500 mg/g)

| INGREDIENTS | % W/W |
|---|---|
| BUPROPION CORE BEAD 600 MG/G | 80 |
| EUDRAGIT ® RS/RL (95%:5%) | 20 |
| PURIFIED WATER | QS |
| TOTAL | 100.00 |

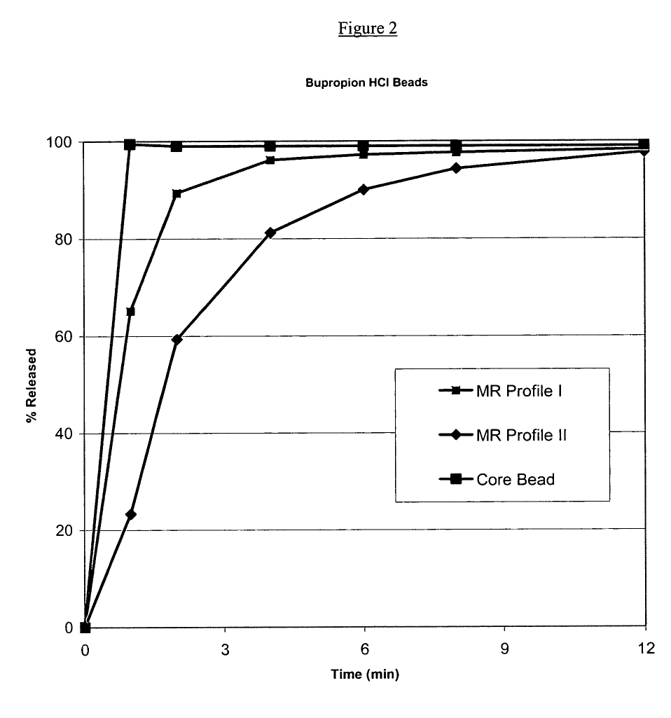

The simulated dissolution profiles of the bupropion core beads (600 mg/g) of Table 9 and the modified release beads of Tables 10 and 11 are shown in FIG. 2.

Tables 12A-12D show the formulation ingredients and weight percent ranges for the manufacture of IR, MRI, MR II and pulsatile formulations.

The beads may be prepared by mixing ingredients in a high shear granulator (Disona, Fluid Air, Chicago, Ill.). The granulated material is extruded with an extruder (Niro, Model E-140, Columbia Md.), and then spheronized into beads using a spheronizer (Niro Model S450, Columbia, Md.). The beads are optionally dried at 50° C. for up to 12 hours. The beads are then coated with seal coating polymer in a fluid bed coater (Glatt AIR, Ramsey, N.J.) at 25 to 30° C.

TABLE 12A
Immediate Release Formulations

| Immediate Release Bupropion (596 mg/g) | mg/dose | mg/g |
|---|---|---|
| Extruded Beads | | |
| BUPROPION HCL | 300 | 595.9 |
| GLYCERYL BEHENATE, NF | 73.1 | 145.4 |
| (COMPRITOL ® 888 ATO) | | |
| MICROCRYSTALLINE CELLULOSE | 33.2 | 66.0 |
| (AVICEL ® PH 101) | | |
| CITRIC ACID, ANHYDROUS | 23.7 | 47.2 |
| BUTYLATED HYDROXY ANISOLE (BHA) | 0.05 | 0.1 |
| SORBITAN MONOOLEATE, NF (SPAN 80) | 57.0 | 113.2 |
| TALC, USP (PHARMA M) | 14.1 | 28.0 |
| Total (extruded beads) | 501 | 995.7 |
| Seal Coating: | | |
| KLUCEL EF | 2.1 | 4.2 |
| BUTYLATED HYDROXY ANISOLE | 0.042 | 0.1 |
| WATER | — | Q.S. |
| Total | 503.1 | 1000.0 |

TABLE 12B
Modified Release Formulation I

| Modified release Formula I (544 mg/g) | mg/dose | mg/g |
|---|---|---|
| Immediate release beads (596 mg/g) | 503.1 | 904.2 |
| EUDRAGIT NE 30D Dispersion (Solids) | 52.1 | 93.7 |
| TALC | 1.2 | 2.1 |
| BUTYLATED HYDROXY ANISOLE | <0.05 | <0.05 |
| WATER | — | Q.S. |
| Total | 556.4 | 1000.0 |

Modified release I beads may be coated using Eudragit suspension in a in a fluid bed coater (Glatt AIR, Ramsey, N.J.) at 25 to 30° C.

TABLE 12C
Modified Release Formulation II

| Modified (Delayed/Extended) | | |
|---|---|---|
| Release Formula II (414 mg/g) | mg/dose | mg/g |
| Modified release I bead (544 mg/g) | 556.4 | 769.2 |
| ACRYLEZE (Solid content) | 167.0 | 230.8 |
| WATER | — | Q.S. |
| Total | 723.4 | 1000.0 |

Modified release II beads are coated using Eudragit suspension in a in a fluid bed coater (Glatt AIR, Ramsey, N.J.) at 30 to 40° C.

TABLE 12D
Pulsatile Formulations

| Two Pulse Formula (300 mg) | mg/dose |
|---|---|
| Immediate Release Bupropion (596 mg/g) | 100.6 mg |
| Modified release Formula II (414 mg/g) | 578.7 mg |
| Total | 679.3 mg |

Two pulse bead containing capsules are prepared by mixing different beads during encapsulation process using a capsule filling machine (MG-2, MG America, Fairfield, N.J.).

### EXAMPLE 4

### Pulsatile Bupropion Capsule Dosage Form

Bupropion core and modified release beads described in Example 3 can be filled into capsules to deliver pulsatile release profiles. For example, predetermined weights of beads can be filled in a capsule using a capsule filling machine (MG-2, MG America, Fairfield, N.J.). The amounts of beads per capsule for a 150 mg strength pulsatile bupropion capsule are shown in Table 13.

TABLE 13
Pulsatile Bupropion Capsule Dosage Formulations

| | | MODIFIED | MODIFIED |
|---|---|---|---|
| | | RELEASE | RELEASE |
| | CORE BEAD | BEAD 1 | BEAD 2 |
| PROFILE | (MG/CAP) | (MG/CAP) | (MG/CAP) |
| SINGLE PULSE | 250 MG | 0 | 0 |
| TWO PULSES | 0 | 137.5 MG | 150 MG |
| THREE PULSES | 0 | 0 | 300 MG |

Capsules containing different amounts of beads of a given strength will generate different dissolution profiles. Also, different dose proportional strengths can be generated by using more beads, such for 75 to 450 mg by the total fill weight.

### EXAMPLE 5

### Pulsatile Escitalopram and Bupropion Capsule Dosage Form

A pulsatile capsule dosage form is prepared by packing a plurality of escitalopram beads and bupropion beads into a capsule. A pulsatile capsule dosage formulation for a 150 mg bupropion/4 mg strength escitalopram capsule is shown in Table 14.

TABLE 14
150 mg Bupropion/4 mg Escitalopram Pulsatile Capsule Dosage Form

| | BUPROPION | BUPROPION | BUPROPION | ESCITALOPRAM | ESCITALOPRAM | ESCITALOPRAM |
|---|---|---|---|---|---|---|
| | CORE PULSE | MR PULSE 1 | MR PULSE 2 | CORE PULSE | MR PULSE 1 | MR PULSE 2 |
| | (MG/CAP) | (MG/CAP) | (MG/CAP) | (MG/CAP) | (MG/CAP) | (MG/CAP) |
| TWO PULSES | 250 MG | 0 | 0 | 25.6 MG | 0 | 0 |
| (ONE PER API) | | | | | | |
| (CAPSULE A) | | | | | | |
| SINGLE MR PULSE | 0 | 0 MG | 300 MG | 0 | 0 MG | 27.2 MG |
| (CAPSULE B) | | | | | | |
| TWO MR PULSES | 0 | 137.5 MG | 150 MG | 0 | 13.2 MG | 13.6 MG |
| (CAPSULE C) | | | | | | |

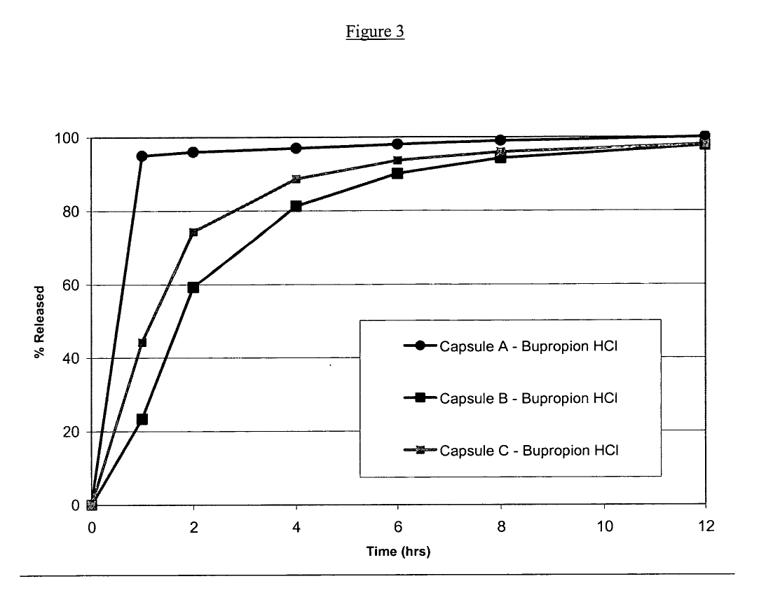

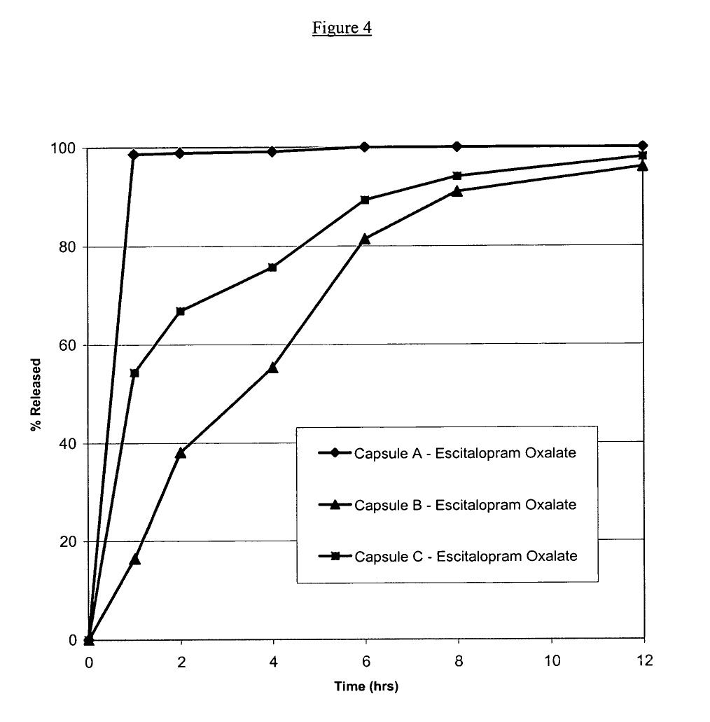

Multiple combinations of beads can be made to meet desired dissolution release profiles. The beads can be filled with a bead blend or multiple hoppers with an encapsulator (such as an MG-2, MG America, Fairfield, N.J.). FIGS. 3 and 4 show the bupropion and escitalopram dissolution rates of the three capsules shown in Table 13, respectively. Dissolution testing for both figures was in accordance with the USP basket method at 100 rpm in 0.1 N HCl.

Dose proportional strengths can be prepared by altering the fill weight.

Formulations for the core and modified release beads that combine bupropion and escitalopram in one system (bead) are susceptible to degradation of both molecules. It was observed that more than 10% loss of potency for each is seen when stored for 1 month at 40° C. and 75% relative humidity. The formulation of the present invention exhibits excellent stability under the same conditions. The beads for both bupropion and escitalopram used in this example exhibit less than 10% loss in potency and in most instances less than 5% loss in potency when stored for 1 month at 40° C. and 75% relative humidity.

Patents, patent applications, publications, product descriptions, and protocols are cited throughout this application, the disclosures of which are incorporated herein by reference in their entireties for all purposes.

What is claimed is:

1: An oral dosage form comprising bupropion or a pharmaceutically acceptable salt thereof and escitalopram or a pharmaceutically acceptable salt thereof.

2: The oral dosage form of claim 1, wherein the bupropion or pharmaceutically acceptable salt thereof and escitalopram or pharmaceutically acceptable salt thereof are physically separated in the oral dosage form.

3: The oral dosage form of claims 1, wherein the oral dosage form comprises from about 50 to about 450 mg of bupropion or a pharmaceutically acceptable salt thereof (calculated based on the weight of a molar equivalent of bupropion hydrochloride).

4: The oral dosage form of claim 1, wherein the oral dosage form comprises from about 75 to about 225 mg of bupropion or a pharmaceutically acceptable salt thereof (calculated based on the weight of a molar equivalent of bupropion hydrochloride).

5: The oral dosage form of claim 1, wherein the oral dosage form comprises 150 mg bupropion or a pharmaceutically acceptable salt thereof.

6: The oral dosage form of claim 1, wherein the oral dosage form comprises 225 mg bupropion or a pharmaceutically acceptable salt thereof.

7: The oral dosage form of claim 1, wherein the oral dosage form comprises bupropion hydrochloride.

8: The oral dosage form of claim 1, wherein the oral dosage form comprises from about 2.5 to about 40 mg escitalopram or a pharmaceutically acceptable salt thereof (calculated based on the weight of a molar equivalent of escitalopram free base).

9: The oral dosage form of claim 1, wherein the oral dosage form comprises 2.5 mg escitalopram or a pharmaceutically acceptable salt thereof.

10: The oral dosage form of claim 1, wherein the oral dosage form comprises 4 mg escitalopram or a pharmaceutically acceptable salt thereof.

11: The oral dosage form of claim 1, wherein the oral dosage form comprises 5 mg escitalopram or a pharmaceutically acceptable salt thereof.

12: The oral dosage form of claim 1, wherein the oral dosage form comprises 10 mg escitalopram or a pharmaceutically acceptable salt thereof.

13: The oral dosage form of claim 1, wherein the oral dosage form comprises 20 mg escitalopram or a pharmaceutically acceptable salt thereof.

14: The oral dosage form of claim 1, wherein the oral dosage form comprises escitalopram oxalate.

15: The oral dosage form of claim 1, wherein the oral dosage form provides immediate release of the bupropion or pharmaceutically acceptable salt thereof.

16: The oral dosage form of claim 1, wherein the oral dosage form provides modified release of the bupropion or pharmaceutically acceptable salt thereof.

17: The oral dosage form of claim 1, wherein the oral dosage form provides immediate release of the escitalopram or pharmaceutically acceptable salt thereof.

18: The oral dosage form of claim 1, wherein the oral dosage form provides modified release of the escitalopram or pharmaceutically acceptable salt thereof.

19: The oral dosage form of claim 1, wherein the oral dosage form, upon ingestion by a patient, induces a statistically significant lower mean fluctuation index for the bupropion or pharmaceutically acceptable salt thereof in the plasma than an immediate release tablet containing the same amount of the bupropion or pharmaceutically acceptable salt thereof, and provides bioavailability of the bupropion or pharmaceutically acceptable salt thereof substantially equivalent to that of three immediate release tablets of the bupropion or pharmaceutically acceptable salt thereof administered one tablet every 6 or more hours, for one day.

20: The oral dosage form of claim 1, wherein less than about 40% of the bupropion or pharmaceutically acceptable salt thereof (based on 100% bupropion or pharmaceutically acceptable salt thereof in the dosage form) is released 2 hours after administration, and more than about 60% of the escitalopram or pharmaceutically acceptable salt thereof is released 12 hours after administration.

21: The oral dosage form of claim 1, wherein the oral dosage form provides pulsated release of the bupropion or pharmaceutically acceptable salt thereof.

22: The oral dosage form of claim 1, wherein the oral dosage form comprises modified release beads of bupropion or a pharmaceutically acceptable salt thereof having at least two different release profiles.

23: The oral dosage form of claim 1, wherein the dosage forms comprises modified release tablets of buproprion or a pharmaceutically acceptable salt thereof.

24: The oral dosage form of claim 1, having an in vitro dissolution profile as measured by the USP Paddle Method at 75 rpm in 900 ml of water at 37° C. such that (i) after 2 hours, less than about 20% by weight of the bupropion or pharmaceutically acceptable salt thereof is released, (ii) after 8 hours, from about 10% to about 60% is released, and (iii) after 24 hours, more than 70% is released.

25: The oral dosage form of claim 1, having an in vitro dissolution profile as measured by the USP Paddle Method at 100 rpm in 900 ml 0.1 N HCl at 37° C. such that (i) after 2 hours, less than about 20% by weight of the bupropion or pharmaceutically acceptable salt thereof is released, (ii) after 8 hours, from about 10% to about 60% is released, and (iii) after 24 hours, more than 70% is released.

26: The oral dosage form of claim 1, wherein the oral dosage form, upon ingestion by a patient, provides a Tₘₐₓ for the escitalopram or pharmaceutically acceptable salt thereof ranging from about 4 to about 35 hours.

27: The oral dosage form of claim 1, wherein the oral dosage form, upon ingestion by a patient, provides a Tₘₐₓ for the escitalopram or pharmaceutically acceptable salt thereof of about 5 hours.

28: The oral dosage form of claim 1, wherein the oral dosage form, upon ingestion by a patient, provides:
(a) a mean Cₘₐₓ for the escitalopram or pharmaceutically acceptable salt thereof that is about 50 to about 85% of that for an immediate release tablet containing the same amount of the escitalopram or pharmaceutically acceptable salt thereof,
(b) a Tₘₐₓ for escitalopram or pharmaceutically acceptable salt thereof of from about 1 to about 8 hours, and
(c) bioavailability for escitalopram or pharmaceutically acceptable salt thereof substantially equivalent to that of an immediate release tablet containing the same amount of the escitalopram or pharmaceutically acceptable salt thereof.

29: The oral dosage form of claim 1, wherein the oral dosage form, upon ingestion by a patient,
(a) induces a statistically significant lower mean fluctuation index in the plasma for the escitalopram or pharmaceutically acceptable salt thereof than an immediate release tablet containing the same amount of the escitalopram or pharmaceutically acceptable salt thereof,
(b) provides a mean minimum plasma concentration (Cₘᵢₙ) for the escitalopram or pharmaceutically acceptable salt thereof substantially equivalent to that of an immediate release tablet containing the same amount of the escitalopram or pharmaceutically acceptable salt thereof,
(c) provides an area under a plasma concentration vs. time curve (AUC) for the escitalopram or pharmaceutically acceptable salt thereof within the range of from about −20% to about +25% of that produced by an immediate release tablet containing the same amount of the escitalopram or pharmaceutically acceptable salt thereof, or
(d) any combination of the foregoing.

30: The oral dosage form of claim 1, wherein the oral dosage form has an AUCO₀₋₂₄ for the escitalopram or pharmaceutically acceptable salt thereof of about 320 to about 400 ng·h/ml.

31: The oral dosage form of claim 1, wherein the oral dosage form has an in vitro dissolution profile as measured by the USP Basket Method at 100 rpm in 900 ml 0.1 N HCl at 37° C. such that after about 30 minutes, more than about 80% by weight of the escitalopram or pharmaceutically acceptable salt thereof is released.

32: The oral dosage form of claim 1, wherein the oral dosage form has an in vitro dissolution profile as measured by the USP Basket Method at 100 rpm in 900 ml 0.1 N HCl at 37° C. such that (i) after 2 hours, from about 10% to about 50% by weight of the escitalopram or pharmaceutically acceptable salt thereof is released, and (ii) after 8 hours, more than about 60% is released.

33: The oral dosage form of claim 1, wherein the oral dosage form comprises 10 mg escitalopram or a pharmaceutically acceptable salt thereof (calculated based on the weight of a molar equivalent of escitalopram free base), and, upon ingestion by a patient, provides a mean maximum plasma concentration (Cₘₐₓ) of the escitalopram or pharmaceutically acceptable salt thereof from about 1 ng/ml to about 50 ng/ml.

34: The oral dosage form of claim 1, wherein the oral dosage form, upon ingestion by a patient, provides a mean maximum plasma concentration (Cₘₐₓ) of the escitalopram or pharmaceutically acceptable salt thereof from about 10 ng/ml to about 18 ng/ml.

35: The oral dosage form of claim 1, wherein the oral dosage form provides a therapeutic effect for at least about 24 hours after administration to a patient.

36: A method of treating a central nervous system disorder in a patient in need thereof comprising administering the oral dosage form of claim 1.

37: The method of claim 36, wherein the oral dosage form is administered once daily.

38: The method of claim 36, wherein the disorder is a mood disorder.

39: The method of claim 38, wherein the mood disorder is major depressive disorder.

40: The method of claim 39, wherein the disorder is an anxiety disorder.

41: A method of treating a sexual dysfunction in a patient in need thereof comprising administering the oral dosage form of claim 1.

42: The method of claim 41, wherein the sexual dysfunction is ejaculation disorder.

43: The method of claim 41, wherein the sexual dysfunction is anorgasmia.

44: The method of claim 41, wherein the sexual dysfunction is decreased libido.

45: A method of treating a patient suffering from treatment resistant depression comprising administering to the patient an effective amount of the oral dosage form of claim 1.
